(19) United States
(12) Patent Application Publication
(10) Pub. No.: US 2023/0172844 A1
(43) Pub. Date: Jun. 8, 2023

(54) CANNABIDIOL ORALLY DISINTEGRATING TABLETS

(71) Applicant: TENSHI KAIZEN PRIVATE LIMITED, Bangalore, Karnataka (IN)

(72) Inventors: Vinod Reddy Bondu KUMAR, Bangalore (IN); Chamarahalli Krishna Sundhar IYER, Bangalore (IN); Venkat IYER, Bangalore (IN)

(73) Assignee: TENSHI KAIZEN PRIVATE LIMITED, Bangalore, Karnataka (IN)

(21) Appl. No.: 17/634,453

(22) PCT Filed: Aug. 12, 2020
(86) PCT No.: PCT/IN2020/050705

§ 371 Date: Feb. 11, 2022

(30) Foreign Application Priority Data
Aug. 12, 2019 (IN) 201941032533

(51) Int. Cl. A61K 9/00; A61K 9/20; A61K 31/047; A61K 9/107; B65B 11/52; B65B 55/00

(52) U.S. Cl. CPC A61K 9/0056; A61K 9/2095; A61K 31/047; A61K 9/2059; A61K 9/2013; A61K 9/2018; A61K 9/2054; A61K 9/1075; B65B 11/52; B65B 55/00

(57) ABSTRACT

The present invention discloses composition comprising an open matrix network carrying pharmaceutically active cannabinoid substance, pharmaceutically acceptable water soluble or water dispersible carrier materials provided as discrete units of the suspension or emulsion in form of liquid units contained in pockets of suitable mould, solid units such as frozen units, gelled units or frozen discrete units, wherein the composition is in the form chosen from sublingual tablet, buccal tablet, and rapidly disintegrating tablet.

### CROSS REFERENCE TO RELATED APPLICATIONS

This application is a U.S. National Stage application of PCT International Patent Application Number PCT/IB2020/050705, which was filed on Aug. 12, 2020, which claims the benefit of priority of the Indian patent application 201941032533, filed on Aug. 12, 2019, the disclosure of all of which is incorporated herein by reference.

### TECHNICAL FIELD OF THE INVENTION

The present invention relates to a pharmaceutical composition comprising an open matrix network carrying pharmaceutically active cannabinoid substance, pharmaceutically acceptable water soluble or water dispersible carrier materials provided as discrete units of the suspension or emulsion in the form of liquid units contained in pockets of suitable mould, solid units such as frozen units, gelled units or frozen discrete units, wherein the composition is in the form chosen from sublingual tablet, buccal tablet, and rapidly disintegrating tablet.

BACKGROUND AND PRIOR ART OF THE INVENTION

Medicinal use of cannabinoids has been known for years or even centuries. However, the psychoactive effects of cannabis and cannabinoids had brought restrictions and controls on its cultivation, trade and use. Lately, the legitimate, medicinal and pharmaceutical benefits of various constituents and derivatives have revived, especially because of lower side effects of pharmaceutical dosage forms derived from Cannabinoids. Use of cannabinoid based pharmaceutical preparations for chronic pain in terminally ill patients and for treatment of muscle spasms are approved in USA. USFDA has granted approval to pharmaceutical active substances (API's) derived from Cannabis sativa. Cannabidiol (CBD), Dronabinol and Nabilon are FDA approved drugs (Marinol, Syndros, Epidiolex) which have received regulatory approvals and products have been launched in the marketfor eg Cannabidiol (also called CBD), which is approved for treatment of seizures, especially for Lennox-Gastaut syndrome and Dravet syndrome. The pharmaceutical use of CBD is most beneficial especially since CBD does not cause intoxication or drug addiction unlike Tetrahydrocannabinol (THC), which is the psycho-potent ingredient of Cannabis. Consequently, the USFDA and USPTO are granting approvals for dosage forms as well as patents and trademarks for these products. Hence, research and development of pharmaceutical formulations and dosage forms of Cannabidiol have been increasing lately.

PCT Publication No. WO2015/065179 discloses a compressed tablet dosage form of Cannabidiol (CBD). US Patent Publication No. 2018/0221332 relates to production of flash melt Cannabis resin and extract oral dosage forms. A method of preparing granulates of cannabinoids with sucrose derivative based granulation liquid is described in U.S. Pat. No. 9,555,019. Oral dosage forms of microgranulate particles of cannabinoids are disclosed in U.S. Pat. No. 9,30,8175. Gelatin matrix pellet based compositions of Cannabinoids including Cannabidiol (CBD) are claimed in US Patent Publication No. 2018/0078504. A method of treatment of focal seizures in Dravet syndrome is the subject matter of invention in US2017/0007551. Oral solution of Cannabidiol is described in US2015/0342902 and US20190167583. The method of treatment of fragile X syndrome is dealt with in U.S. Pat. No. 10,213,390.

US2016/0058866 describes compositions comprising at least one of PVP/vinyl acetate co-polymer forming open matrix network and at least one tetrahydrocannabinol and cannabidiol in the said matrix. While this specification refers to the option of lyophilisation, the process thereof or the detailed compositions for such dosage forms are not specifically disclosed or enabled to practice. The said US Patent Publication No. 2016/0058866 does not disclose the lyophilisation process nor provides any specific examples describing the process. Further, the said prior art disclosure is relating to Tetrahydrocannabinol and Cannabidiol which have contradictory therapeutic benefits.

A significant proportion of patients have difficulty swallowing (dysphagia) in the acute phase, and many have ongoing problems. This potentially can lead to a reduction in patient compliance when such patients are administered oral formulations that must be swallowed intact. Other patients may also suffer from dysphagia as it is common among all age groups and is observed in about 35% of the general population, as well as up to 60% of the elderly institutionalized population and about 20% of all patients in long-term care facilities.

In case of tablets that disintegrate inside the mouth, drugs may be absorbed in the buccal, pharyngeal, and gastric regions. This possibly may facilitate rapid drug therapy intervention and increased bioavailability of drugs. Because the pre-gastric drug absorption avoids the first-pass metabolism, the drug dose can be reduced if a significant amount of the drug is lost through the hepatic metabolism. Further, erratic absorption, poor bioavailability and a lasting need to achieve satisfactory or therapeutic plasma level, with fast onset of drug action are the drawbacks of administering cannabinoids.

Having felt the need for ORALLY disintegrating dosage form of therapeutically significant component, Cannabidiol (CBD), the present inventors have come up with a detailed process and specific compositions for ORALLY disintegrating tablet dosage forms of Cannabidiol (CBD), prepared by a process of lyophilisation, wherein the discrete units are obtained by freeze drying of frozen liquid/suspension filled blisters and packed under cGMP conditions and wherein said dosage form disintegrates in the mouth in less than 10 seconds.

### SUMMARY OF THE INVENTION

One aspect of the present invention provides compositions which comprise a) cannabinoid in a concentration ranging from 1% to 50% by weight of the composition, b) binding/matrix forming agent in a concentration ranging from 0.2% to 12% by weight of the composition, c) structure forming agent in a concentration ranging from 2% to 10% by weight of the composition, d) emulsifying agent or oleaginous vehicle in a concentration ranging from 0.1.to 20%; e) solubilizing agent in a concentration ranging from 0 to 25% and other pharmaceutically acceptable excipients.

The second aspect of the invention provides a process for the preparation of rapidly disintegrating freeze dried solid oral tablet composition comprising cannabinoid comprising:
(i) preparing a suspension comprising cannabidiol, an emulsifying/oleaginous carrier, matrix forming agent/binder(s), structure-forming agent(a); solubilizing agent and other pharmaceutically acceptable ingredients in a solvent to obtain a homogenous suspension,
(ii) transferring the homogenous suspension to a dosing tank and stirring/homogenising the said suspension until the end of the filling process,
(iii) Forming blister pockets in blister filling machine,
(iv) Using peristaltic pump or any pump of equivalent performance to dose accurate amount of suspension into preformed blister pockets,
(v) freezing the drug suspension filled blister pockets by passing through liquid nitrogen freezing tunnel temperature ranging from −160° C. to −70° C. and pressure ranging from 3 kg/cm² to 9 kg/cm² to obtain frozen products,
(vi) placing the frozen products into lyophilizer,
(vii) lyophilizing the said frozen blister sheets at a primary drying temperature ranging from −30° C. to 0° C. followed by lyophilizing at a secondary drying temperature ranging from 15° C. to 40° C. to obtain a rapidly disintegrating solid oral tablet composition comprising cannabidiol.

In the third aspect, the invention provides compositions that exhibit rapid dispersion of the Cannabinoid (e.g. in the form of granules) in physiological solutions (e.g. saliva). This rapid dispersion facilitates the swallowing of the drug by the patient. The dissolution rate of the cannabinoid should also be maintained within acceptable parameters. Tablets are typically considered to have a suitable dissolution profile if they comply with the requirement: Q=70% after 45 minutes. Suitable conditions for such measurements according to the European Pharmacopoeia include Apparatus 2, paddle speed 75 rpm, dissolution medium 900 mL with 0.2% (w/v) Tween 80.

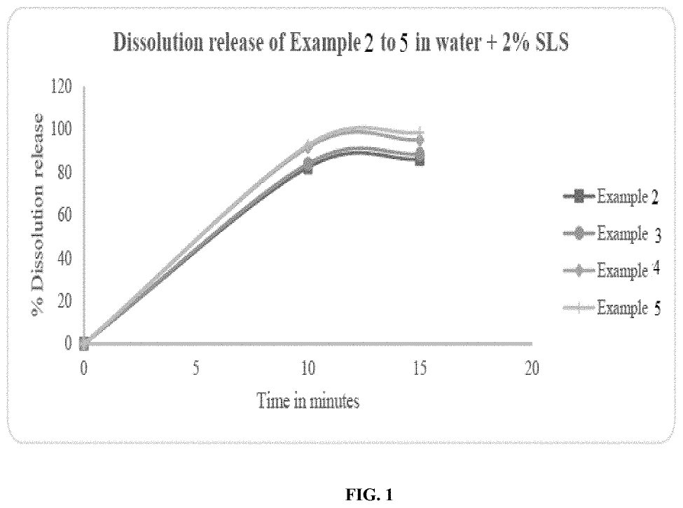

In a further embodiment, the comparative dissolution profiles of the tablet compositions namely in examples 2 to 5, as shown in FIG. 1 is provided such that Q=70% after 45 minutes.

In a further embodiment, the invention provides stability studies of the freeze dried compositions prepared in accordance with the examples 8 and 10, wherein, the blister packaged tablets were stored under different storage conditions of 20° C.±5° C. and 40° C. for 3 months. The tablets were found to be stable under these storage conditions, with no detection of unspecified impurities and with Cannabinoid content after storage were well within limits (THC component BQL).

DESCRIPTION OF DRAWING

FIG. 1 shows comparative dissolution profiles of the tablet compositions of examples 2 to 5. The freeze-dried tablets disintegration resulted in 5 to 15 seconds (limit NMT 30 seconds). The dissolution release of the samples in water with 2% SLS was found to have 80 Q release in 3 to 15 minutes with % RSD NMT 5%.

### DETAILED DESCRIPTION OF THE INVENTION

The invention will now be described in detail in connection with certain preferred and optional embodiments, so that various aspects thereof may be more fully understood and appreciated.

The present invention provides Rapidly disintegrating (RDT) freeze dried tablet compositions comprising Cannabinoids that overcomes the problem of compliance for people who have dysphagia or gastric reflex issues.

The term ‘lyophilized’ and ‘freeze dried’ are used alternately in the entire specification; which are synonymous to each other and as such the skilled person appreciate the same.

Similarly, the term ‘tablet dosage form’ and ‘tablet compositions’ are used alternately in the entire specification; which are synonymous to each other and as such the skilled person appreciate the same.

Dosage Form (DF) is defined as the physical form of a dose of a chemical compound used as a drug or medication intended for administration or consumption.

The present invention discloses compositions of rapidly disintegrating solid oral tablet dosage forms which comprises an open matrix network carrying the pharmaceutically active substance such as cannabidiol or cannabinoid along with pharmaceutically acceptable excipients and carrier solvents such as water with or without co-solvent such as alcohol, wherein the composition filled blisters are directly freeze dried and sealed to form ready to dispatch packs and wherein said dosage form disintegrates in the mouth in less than 10 seconds.

Accordingly, in an embodiment, the invention provides rapidly disintegrating freeze dried tablet composition which comprises a) cannabinoid in a concentration ranging from 1% to 50% by weight of the composition, b) binding/matrix forming agent in a concentration ranging from 0.2% to 12% by weight of the composition, c) structure forming agent in a concentration ranging from 2% to 10% by weight of the composition, d) emulsifying agent or oleaginous vehicle in a concentration ranging from 0.1.to 20%.

The tablet composition according to the invention optionally comprise a solubilizing agent in a concentration ranging from 0 to 25% and other pharmaceutically acceptable excipients.

The other pharmaceutical excipients are selected from the group consisting of diluents, bulking agent, adhesive agents, emollients, surfactant polymers, colorants, sweeteners, flavouring agents, taste-masking agents or preservatives.

In a preferred embodiment, the present invention provides a process for preparation of rapidly disintegrating freeze dried solid oral tablet composition comprising cannabinoid comprising;
(i) preparing a suspension comprising cannabidiol, an emulsifying/oleaginous carrier, matrix forming agent/binder(s), structure-forming agent(a); solubilizing agent and other pharmaceutically acceptable ingredients, in a solvent to obtain a homogenous suspension,
(ii) transferring the homogenous suspension to a dosing tank and stirring/homogenising the said suspension until the end of the filling process,
(iii) Forming blister pockets in blister filling machine,
(iv) Using peristaltic pump (or any pump of equivalent performance) to dose accurate amount of suspension into preformed blister pockets,
(v) freezing the drug suspension filled blister pockets by passing through liquid nitrogen freezing tunnel temperature ranging from −160° C. to −70° C. and pressure ranging from 3 kg/cm² to 9 kg/cm² to obtain frozen products,
(vi) placing the frozen products into lyophilizer,
(vii) lyophilizing the said frozen blister sheets at a primary drying temperature ranging from −30° C. to 0° C. followed by lyophilizing at a secondary drying temperature ranging from 15° C. to 40° C. to obtain a rapidly disintegrating solid oral tablet composition comprising cannabidiol.
   Accordingly, the present invention provides lyophilized, solid oral rapidly disintegrating tablet dosage forms comprising a cannabinoid or a derivative thereof with an oleaginous vehicle, emulsifying agent(s), matrix-forming agent(s) or binding agent(s); solubilizing agent and structure-forming agent(s), along with other pharmaceutical excipients/carriers.

The pharmaceutically active ingredient, i.e., cannabinoid, as used in the composition of the present invention may be selected from synthetic, semi synthetic or natural cannabinoid and extract of a cannabis plant, derivatives of cannabinoids and combination of cannabis plant constituents.

Accordingly, the cannabinoid comprise of at least one of the but are not limited to Cannabidiol (CBD), delta-9-tetrahydrocannabinol (THC), Cannabinol (CBN), Cannabigerol (CBG), Cannabichromene (CBC), Cannabicyclol (CBL), Cannabivarin (CBV), Tetrahydrocannabivarin (THCV), Cannabidivarin (CBVD), Cannabichromevarin (CBVC), Cannabigerovarin (CBVG), Cannabigerol monoethyl ether (CBGM), a derivative, an acid form, a precursor, and a combination thereof. The active ingredient may be present in the solid form, powder of crystal form, oil form, taste masked form, enteric or a controlled release form.

The matrix forming agent(s) or the binding agent is selected from the group comprising fish gelatine sodium alginate, pullulan, maltodextrin, sodium alginate, xanthan gum, and hypromellose.

The oleaginous vehicle or the emulsifying or the/dispersing agent is selected from the group comprising olive oil, sesame oil, castor oil, coconut oil corn oil, lecithin, isolecithin, glycine, polysorbate 20 or polysorbate 80.

The structure forming agent is either mannitol or dextran.

The composition optionally includes a solubilizing agent selected from the group comprising betacyclodextrin, Hydroxypropyl Beta-cyclodextrin or sufobutyl betacyclodextrin.

Additionally, the process of homogenization performed in step (ii) of the process described in the present invention is to ensure and maintain the uniform homogeneity of the suspension up to the end of the filling process.

In an embodiment, the present invention provides a process for producing a lyophilized rapidly disintegrating solid oral dosage comprising cannabidiol, the said process comprising;
(i) preparing a suspension comprising cannabidiol, an oleaginous carrier, hypromellose and other pharmaceutically acceptable ingredients in a solvent to obtain a homogenous suspension,
(ii) transferring the homogenous suspension to a dosing tank and stirring/homogenising the said suspension until the end of the filling process,
(iii) forming blister pockets in blister forming machine,
(iv) Using peristaltic pump (or any pump of equivalent performance) to dose accurate amount of suspension into preformed blister pockets,
(v) freezing the drug suspension filled blister pockets by passing through liquid nitrogen freezing tunnel at a temperature ranging from −160° C. to −70° C. and pressure ranging from 5 kg/cm² to 7 kg/cm²,
(vi) placing the frozen product into lyophilizer and lyophilizing the said blister sheets at a primary drying temperature ranging from −35° C. to −5° C. followed by lyophilizing at a secondary drying temperature ranging from 20° C. to 40° C. to obtain a rapidly disintegrating solid oral dosage form comprising cannabidiol.

In accordance with the aforesaid embodiment, the present invention provides a composition comprising a lyophilized oral solid rapidly disintegrating dosages form in an oil-water emulsion, the said composition comprising Cannabidiol with a oleaginous vehicle selected from the group comprising of olive oil, sesame oil, castor oil, coconut oil, corn oil, along with pharmaceutically acceptable excipients selected from the group comprising emulsifying agent, matrix forming agent or binding agent and structure forming agent.

In another embodiment, the present invention provides a process for producing a lyophilized rapidly disintegrating solid oral tablet composition comprising cannabidiol, the said process comprising;
(i) preparing a suspension comprising cannabidiol as an oil, hypromellose and other pharmaceutically acceptable ingredients in a solvent to obtain a homogenous suspension,
(ii) transferring the homogenous suspension to a dosing tank and stirring/homogenising the said suspension until the end of the filling process
(iii) Forming blister pockets in blister forming machine
(iv) Using Peristaltic pump (or any pump of equivalent performance) to dose accurate amount of suspension into preformed blister pockets.
(v) freezing the drug suspension filled blister pockets by passing through liquid nitrogen freezing tunnel at a temperature ranging from −160° C. to −70° C. and pressure ranging from 5 kg/cm² to 7 kg/cm²,
(vi) placing the frozen blister sheets containing product into a lyophilizer and
(vii) Lyophilizing the said frozen blister pockets at a primary drying temperature ranging from −35° C. to −5° C. followed by lyophilizing at a secondary drying temperature ranging from 20° C. to 40° C. to obtain a rapidly disintegrating solid oral tablet comprising cannabidiol.

In accordance with the aforesaid embodiment, the present invention provides a composition comprising solid oral, rapidly disintegrating tablet dosage form comprising cannabidiol as an oil in an aqueous suspension comprising an emulsifying agent, matrix forming agent or binding agent and structure forming agent.

In yet another embodiment, the present invention provides a process for producing a lyophilized rapidly disintegrating solid oral tablet dosage form comprising cannabidiol, the said process comprising;
(i) preparing a suspension comprising cannabidiol, pullulan and other pharmaceutically acceptable ingredients in a solvent to obtain a homogenous suspension,
(ii) transferring the homogenous suspension to a dosing tank and stirring/homogenising the said suspension until the end of the filling process,
(iii) forming blister pockets in blister forming machine,
(iv) Using Peristaltic pump (or any pump of equivalent performance) to dose accurate amount of suspension into preformed blister pockets.
(v) freezing the drug suspension filled blister pockets by passing through liquid nitrogen freezing tunnel at a temperature ranging from −160° C. to −70° C. and pressure ranging from 5 kg/cm² to 7 kg/cm²,
(vi) placing the frozen blister sheets containing product in to a lyophilizer and
(vii) Lyophilizing the said frozen blister pockets at a primary drying temperature ranging from −35° C. to −5° C. followed by lyophilizing at a secondary drying temperature ranging from 20° C. to 40° C. to obtain a rapidly disintegrating solid oral tablet dosage units comprising cannabidiol.

In accordance with the aforesaid embodiment, the present invention provides a rapidly disintegrating freeze dried tablet composition comprising pullulan as a matrix forming or binding agent, a dispersing agent and a structure forming agent.

In a further embodiment, the present invention provides a process for producing a rapidly disintegrating freeze dried tablet composition comprising cannabidiol, the said process comprising;
(i) preparing a suspension comprising cannabidiol, sodium alginate and other pharmaceutically acceptable ingredients in a solvent to obtain an homogenous suspension,
(ii) transferring the homogenous suspension to a dosing tank and stirring/homogenising the said suspension until the end of the filling process
(iii) forming blister pockets in blister forming machine
(iv) using Peristaltic pump (or any pump of equivalent performance) to dose accurate amount of suspension into preformed blister pockets.
(v) freezing the drug suspension filled blister pockets by passing through liquid nitrogen freezing tunnel at a temperature ranging from −160° C. to −70° C. and pressure ranging from 5 kg/cm² to 7 kg/cm²
(vi) placing the frozen blister sheets containing product into a lyophilizer and
(vii) Lyophilizing the said frozen blister pockets at a primary drying temperature ranging from −35° C. to −5° C. followed by lyophilizing at a secondary drying temperature ranging from 20° C. to 40° C. to obtain a rapidly disintegrating solid oral tablet units comprising cannabidiol.

The aforesaid process results in the formation of a lyophilized, oral, solid, rapidly disintegrating tablets comprising cannabidiol, sodium alginate as the matrix forming agent, a dispersing agent and a structure forming agent for rapid dispersion.

In one embodiment, the present invention provides a process for producing a lyophilized, rapidly disintegrating solid oral tablets comprising cannabidiol, the said process comprising;
(i) preparing a suspension comprising cannabidiol, fish gelatine and other pharmaceutically acceptable ingredients in a solvent to obtain a homogenous suspension,
(ii) transferring the homogenous suspension to a dosing tank and stirring/homogenising the said suspension until the end of the filling process,
(iii) forming blister pockets in blister forming machine,
(iv) using Peristaltic pump (or any pump of equivalent performance) to dose accurate amount of suspension into preformed blister pockets.
(v) freezing the drug suspension filled blister pockets by passing through liquid nitrogen freezing tunnel at a temperature ranging from −160° C. to −70° C. and pressure ranging from 5 kg/cm² to 7 kg/cm²
(vi) placing the frozen blister sheets containing product in to a lyophilizer, and
(vii) Lyophilizing the said frozen blister pockets at a primary drying temperature ranging from −35° C. to −5° C. followed by lyophilizing at a secondary drying temperature ranging from 20° C. to 40° C. to obtain a rapidly disintegrating solid oral tablet units comprising cannabidiol.

The aforesaid process results in the formation of a lyophilized, oral, solid, rapidly disintegrating tablets comprising cannabidiol, fish gelatine as the matrix forming agent, a dispersing agent and a structure forming agent for rapid dispersion.

The discrete units of the suspension or solution or emulsion may be in the form of liquid units or suspension units, for example contained within the pockets of a suitable mould, solid units, for example frozen units, or gelled units where the carrier material readily forms a gel.

The liquid solution or suspension which may be contained within the pockets of a suitable mould is frozen, for example by passing a gaseous cooling medium, such as Liquid Nitrogen over the mould, or by inserting the mould into a nitrogen spray freezing chamber, or cooling by passing the mould over a cold surface using Liquid Nitrogen. After the dosage forms have been frozen, the mould is loaded into the Freeze drier/Lyophiliser for Freeze drying or may be stored in a cold storage equipment before loading into the Freeze Drier/Lyophiliser, prior to drying.

The solid dosage forms are prepared by the sublimation or removal of solvent/water from a solution or suspension or emulsion comprising the pharmaceutically active substance and the carrier material. Sublimation or removal of solvent or water is carried out by freeze drying. The solvent or water is sublimed in a freeze-drying process under a reduced pressure which transforms the solid solvent directly into a vapour. The freeze-drying process will generally be carried out in a freeze-drying chamber typically operating under a vacuum of 0.1 to 1.0 mBar for a period from 100 to 800 minutes.

The process of the present invention is also intended to be applied to pharmaceutically acceptable salt form of the active ingredient or the medicament.

The other excipients are selected from the group consisting of diluents such as microcrystalline cellulose, bulking agents such as lactose monohydrate, adhesive agents such as potato starch and amylopectin, emollients, surfactant polymers such as Macrogols, colorants, sweeteners, flavouring agents, taste-masking agents or preservatives can be added to the aqueous phase of the present composition.

The process of taste masking can be carried out by any of the processes known in the art, not limiting to complexation with cyclodextrins, ion exchange resins or any other suitable agents. Taste masking can also be achieved by coating the solid pharmaceutical dosage forms with water soluble or insoluble polymers or polymers having pH dependent solubility or waxes.

The solvent used in forming the solution or suspension of pharmaceutically active substance is preferably purified water, but it may be admixed with co-solvent such as alcohol, if it desired to improve the solubility of active substance.

The specific examples of the compositions and processes thereof are described hereinafter. The following examples given by way of illustration will serve to illustrate the practice of this invention and therefore should not be construed to limit the scope of the invention.

### EXAMPLE 1

| S. No. | Ingredients | Functional category | % Composition |
|---|---|---|---|
| 01 | Cannabidiol (Solid form) | Active | 4.00 |
| 02 | Maltodextrin | Matrix former (Binder) | 0.40-6.00 |
| 03 | Hypromellose | Matrix former (Binder) | 0.40-6.00 |
| 04 | Mannitol | Structure forming agent | 2.00-10.00 |
| | | (Diluent) | |
| 05 | Olive oil/Sesame oil | Oleaginous vehicle | 2.00-20.00 |
| 06 | Polysorbate 80 | Emulsifying agents | 0.05-1.50 |
| 07 | Aspartame | Sweetener | 0.20-0.60 |
| 08 | Flavour | Flavouring Agent | 0.40-2.40 |
| 09 | Purified water * | Carrier (Vehicle) | 90.55-49.50 |

Note
* Water was removed during processing

Manufacturing Process:

The ingredients cannabidiol, maltodextrin, hypromellose, mannitol, olive oil, polysorbate 80, aspartame and flavour were accurately weighed and dispensed. Oil in water emulsion was prepared using the following procedure.

Step 1: A weighed quantity of purified water was transferred into a beaker and the stirrer was placed in the centre of the beaker and stirred at 500 rpm.

Note: Each i from step 2 to step 6 was added under steady stirring maintained between 200 to 4000 rpm and homogenising at 1000-10,000 rpm.

Step 2: Added slowly, the weighed quantity of maltodextrine to beaker under continuous stirring and homogenisation

Step 3: Added weighed quantity of hypromellose to the solution of step 2 and stirred/homogenised well until a clear solution was obtained.

Step 4: Added weighed quantity of mannitol to the solution of step 3 and stirred/homogenised well until a clear solution was obtained.

Step 5: The weighed quantity of aspartame was added to the suspension of step 4 and stirred/homogenised well until a uniform suspension was obtained.

Step 6: The weighed quantity of flavour was added to suspension of step 5 and stirred/homogenised well until a uniform suspension was obtained.

Step 7: In separate vessel added weighed quantity of cannabidiol to olive oil and mixed/homogenised well until a uniform mucilage was obtained.

Step 8: The weighed quantity of polysorbate 80 was added to mucilage of step 7 with continuous stirring and homogenisation.

Step 9: The above step 7 mucilage was added drop by drop with continuous mixing and homogenising to step 6 suspension until to obtain homogenous emulsion obtained.

Filling and Loading

Transferred the emulsion obtained from step 9 to dosing tank with assembly connected with peristaltic pump. The above suspension was kept under stirring at 300 rpm to 1000 rpm and homogenising at 1000-5000 rpm until the end of the filling process.

Quick Freezing

Ran the suspension filled blisters sheets through a liquid nitrogen freezing tunnel with targeted liquid nitrogen tunnel temperature −110±40° C. & pressure variations from 5-7 Kg/cm²

Lyophilisation

After loading required quantity of frozen blister sheets, the door of lyophilizer is closed and the product was lyophilized as per the below lyo cycle parameters.

Lyo Cycle Parameters—

Primary drying temperature −35 to −5° C., Secondary drying temperature 20° C. to 40° C.

Unloading: After completion of Lyo cycle, the blisters are unloaded from the lyophilizer carefully.

Sealing: The unloaded blisters were sealed with paper aluminium lidding foil with sealing temperature range of 180±30° C. to meet leak test pass criteria.

### EXAMPLE 2

| S. No. | Ingredients | Functional category | % Composition |
|---|---|---|---|
| 01 | Cannabidiol (isolate) | Active | 6.70 |
| 02 | Hydroxypropyl Beta- | Solubilizing agent | 3.30-20.00 |
| | cyclodextrin | | |
| 03 | Mannitol | Structure forming agent | 3.00-6.00 |
| | | (Diluent) | |
| 04 | Pullulan | Matrix forming agent | 1.00-3.00 |
| 04 | Aspartame | Sweetener | 0.50-2.00 |
| 05 | Polysorbate 80 | Emulsifying agent | 0.5-2.00 |
| 06 | Flavour | Flavouring agent | 0.5-2.00 |
| 07 | Purified water * | Carrier (Vehicle) | 60.00-80.00 |

* Water was removed during lyophilization processing

### EXAMPLE 3

| S. No. | Ingredients | Functional category | % Composition |
|---|---|---|---|
| 01 | Cannabidiol (isolate) | Active | 6.70 |
| 02 | Beta-cyclodextrin | Solubilizing agent | 3.30-20.00 |
| 03 | Mannitol | Structure forming agent | 3.00-6.00 |
| | | (Diluent) | |
| 04 | Pullulan | Matrix forming agent | 1.00-3.00 |
| 04 | Aspartame | Sweetener | 0.50-2.00 |
| 05 | Polysorbate 80 | Emulsifying agent | 0.5-2.00 |
| 06 | Flavour | Flavouring agent | 0.5-2.00 |
| 07 | Purified water * | Carrier (Vehicle) | 60.00-80.00 |

* Water was removed during lyophilization processing

Manufacturing Process for Example 2 and 3:

(i) preparing a complex suspension comprising Cannabidiol, Hydroxypropyl betacyclodextrin/beta-cyclodextrin (std.), an oleaginous carrier, binder(s), matrix-forming agent(a) and pharmaceutically acceptable ingredients to obtain a homogenous suspension,

(ii) transferring the homogenous suspension to a dosing tank and stirring/homogenizing the said suspension until the end of the filling process

(iii) Forming blister pockets in blister filling machine, following by dosing the suspension accurately into preformed blister pockets by using peristaltic pump (or any pump of equivalent performance)

(iv) freezing the drug suspension filled blister pockets by passing through liquid nitrogen freezing tunnel temperature ranging from −70° C. to −160° C. and pressure ranging from 3 kg/cm2 to 9 kg/cm2,

(v) placing the frozen product into lyophilizer and Lyophilizing the said frozen blister sheets at a primary drying temperature ranging from −30° C. to 0° C. followed by lyophilizing at a secondary drying temperature ranging from 15° C. to 20° C. under specific ranges of vacuum to obtain a rapidly disintegrating solid oral/sublingual dosage form comprising Cannabidiol.

The freeze-dried tablets thus obtained were analysed for complete finished product specification (Description, identification, assay, related substance, dissolution and water content). The freeze-dried tablets disintegration resulted in 5 to 15 seconds (limit NMT 30 seconds). The dissolution release of the samples in water with 2% SLS was found to have 80 Q in 3 to 15 minutes with % RSD NMT 5%.

### EXAMPLE 4

| S. No. | Ingredients | Functional category | % Composition |
|---|---|---|---|
| 01 | Cannabidiol (isolate) | Active | 12.50 |
| 02 | Hydroxypropyl Beta- | Solubilizing agent | 6.25-25.00 |
| | cyclodextrin | | |
| 03 | Mannitol | Structure forming agent | 4.00-6.00 |
| | | (Diluent) | |
| 04 | Pullulan | Matrix forming agent | 1.00-3.00 |
| 04 | Aspartame | Sweetener | 0.50-2.00 |
| 05 | Polysorbate 80 | Emulsifying agent | 0.5-2.00 |
| 06 | Flavour | Flavouring agent | 0.5-2.00 |
| 07 | Purified water * | Carrier (Vehicle) | 45.00-60.00 |

* Water was removed during lyophilization processing

### EXAMPLE 5

| S. No. | Ingredients | Functional category | % Composition |
|---|---|---|---|
| 01 | Cannabidiol (isolate) | Active | 12.50 |
| 02 | Beta-cyclodextrin | Solubilizing agent | 6.25-25.00 |
| 03 | Mannitol | Structure forming agent | 4.00-6.00 |
| | | (Diluent) | |
| 04 | Pullulan | Matrix forming agent | 1.00-3.00 |
| 04 | Aspartame | Sweetener | 0.50-2.00 |
| 05 | Polysorbate 80 | Emulsifying agent | 0.5-2.00 |
| 06 | Flavour | Flavouring agent | 0.5-2.00 |
| 07 | Purified water * | Carrier (Vehicle) | 45.00-60.00 |

* Water was removed during lyophilization processing

Manufacturing Process for Example 4 and 5:

(i) preparing a complex suspension comprising Cannabidiol, Hydroxypropyl betacyclodextrin/beta-cyclodextrin (std.), an oleaginous carrier, binder(s), matrix-forming agent(a) and pharmaceutically acceptable ingredients to obtain a homogenous suspension,

(ii) transferring the homogenous suspension to a dosing tank and stirring/homogenizing the said suspension until the end of the filling process

(iii) Forming blister pockets in blister filling machine, following by dosing the suspension accurately into preformed blister pockets by using peristaltic pump (or any pump of equivalent performance)

(iv) freezing the drug suspension filled blister pockets by passing through liquid nitrogen freezing tunnel temperature ranging from −160° C. to −70° C. and pressure ranging from 3 kg/cm2 to 9 kg/cm2,

(v) placing the frozen product into lyophilizer and Lyophilizing the said frozen blister sheets at a primary drying temperature ranging from −30° C. to 0° C. followed by lyophilizing at a secondary drying temperature ranging from 15° C. to 20° C. under specific ranges of vacuum to obtain a rapidly disintegrating solid oral/sublingual dosage form comprising Cannabidiol.

The freeze-dried tablets thus obtained were analysed for complete finished product specification (Description, identification, assay, related substance, dissolution and water content). The freeze-dried tablets disintegration resulted in 5 to 15 seconds (limit NMT 30 seconds). The dissolution release of the samples in water with 2% SLS was found to have 80 Q in 3 to 15 minutes with % RSD NMT 5%.

The comparative dissolution profiles of examples 7 to 10 is shown in FIG. 1.

Stability Study

The final composition was packaged in commercial packing blisters. These packaged tablets were stored under different storage conditions of 20° C.±5° C. and 40° C. for 3 months, and the stability data for examples 3 and 5 is provided herein below. The tablets were found to be stable under these storage conditions with no detection of unspecified impurities and with CBD content after storage were well within limits (THC component BQL).

The stability data for examples 3 and 5 is shown in below table.

| | Example 3 | | | | | | Example 5 | | | | | |
|---|---|---|---|---|---|---|---|---|---|---|---|---|
| | 40° C./ | | 25° C./ | | 30° C./ | | 40° C./ | | 25° C./ | | 30° C./ | |
| | 75% RH | | 60% RH | | 65% RH | | 75% RH | | 60% RH | | 65% RH | |
| Impurity | Initial | 3M | Initial | 3M | Initial | 3M | Initial | 3M | Initial | 3M | Initial | 3M |
| Individual | ND | ND | ND | ND | ND | ND | ND | ND | ND | ND | ND | ND |
| Unspecified | | | | | | | | | | | | |
| % Total | 0.9 | 0.8 | 1 | 0.9 | 1.0 | 0.9 | 0.8 | 0.8 | 0.8 | 0.8 | 0.8 | 0.8 |
| Impurities | | | | | | | | | | | | |

M—Months

### EXAMPLE—6

CBD RDT 20 mg (lyophilized sublingual tablet administration)

| S. No. | Ingredients | Functional category | % Composition |
|---|---|---|---|
| 01 | Cannabidiol (isolate) | Active | 1.43 to 4.3 |
| 02 | Beta-cyclodextrin | Solubilizing agent | 5-20 |
| 03 | Microcrystalline | Diluent/binding agent | 1-2 |
| | cellulose | | |
| 04 | Lactose monohydrate | Bulking agent | 4-8 |
| 05 | Pototata starch and | Adhesive agents | 2-5 |
| | Amylopectin | | |
| 06 | Glycine | Emollient/emulsifying | 0.25-1 |
| | | agent | |
| 07 | Macrogol | Surfactant polymer | 0.25-1 |
| 08 | Mannitol | Structure forming agent | 5.00-20.00 |
| | | (Diluent) | |
| 09 | Pullulan | Matrix forming agent | 2.00-5.00 |
| 10 | Aspartame | Sweetener | 0.50-2.00 |
| 11 | Polysorbate 80 | Emulsifying agent | 0.1-1.00 |
| 12 | Flavour | Flavouring agent | 0.1-3.00 |
| 13 | Purified water * | Carrier (Vehicle) | 45.00-60.00 |

* Water was removed during lyophilization processing

The Manufacturing Process for Example 6:

(i) Preparing the solution of Potato starch and Amylopectin, an adhesive agent by adding in purified water and mixing thoroughly with a stirrer. Then heating the mixture to 40° C. to 50° C. for 10 to 30 minutes to allow dissolving of the polymers.

(ii) Once the solution cooled down to room temperature, adding a surfactant polymer, emulsifying agent, diluent, bulking agent, matrix-forming agent and structure-forming agent i.e. Macrogol, glycine, polysorbate 80, microcrystalline cellulose, lactose, Pullulan and mannitol individually, under stirring to obtain a homogenous solution.

(iii) Preparing a complex suspension by adding Cannabidiol, Hydroxypropyl betacyclodextrin/Beta-cyclodextrin (std.), a solubilizing agent, Sweetener and Flavouring agent to above solution to obtain a homogenous suspension

(iv)Transferring the homogenous suspension to a dosing tank and stirring/homogenizing the said suspension until the end of the filling process

(v) Forming blister pockets in blister filling machine. Use of peristaltic pump (or any pump of equivalent performance) to dose accurate amount of suspension into preformed blister pockets

(vi) Freezing the drug suspension filled blister pockets by passing through liquid nitrogen freezing tunnel temperature ranging from −160° C. to −70° C. and pressure ranging from 3 kg/cm2 to 9 kg/cm2,

(vii) The frozen product is then placed into lyophilizer. Lyophilizing the said frozen blister sheets at a primary drying temperature ranging from −30° C. to 0° C. followed by lyophilizing at a secondary drying temperature ranging from 15° C. to 20° C. under specific ranges of vacuum to obtain a rapidly disintegrating solid oral/sublingual dosage form comprising Cannabidiol.

(viii) Analyze the freeze-dried tablets for complete finished product specification (Description, identification, assay, related substance, dissolution and water content).

### EXAMPLE—7

Qualitative and Quantitative Composition CBD Nanoemulsion RDT 10 mg

| S. No. | Ingredients | Functional category | % Composition |
|---|---|---|---|
| 01 | Cannabidiol | Active | 1.0 to 5.0 |
| 02 | Polysorbate 80 | Emulsifying agent | 0.5-4.00 |
| 03 | Lecithin | Emulsifier | 0.5 to 2.0 |
| 04 | Purified water | Carrier vehicle | 80-90 |

Bulk suspension preparation

| | | | |
|---|---|---|---|
| 05 | Pullulan | Matrix forming agent | 1.00-5.00 |
| 06 | Mannitol | Structure forming agent | 2.00-10.00 |
| 07 | Aspartame | Sweetener | 0.50-2.00 |
| 08 | Polysorbate 80 | Emulsifying agent | 0.5-1.5 |
| 09 | Flavour | Flavouring agent | 0.5-1.5 |
| 10 | Purified water * | Carrier (Vehicle) | 15.00-20.00 |

* Water was removed during lyophilization processing

Manufacturing Process:

Nano Emulsion preparation:

Oil in water emulsion preparation procedure.

Polysorbate 80 and Cannabidiol oil were taken in a separate beaker and subjected to sonification using ultrasonicator. This mixture was slowly added to the Lecithin/water pre-mix placed on water bath under sonification for 10 minutes at 30%-90% amplitude or high-pressure homogenizer to form a Nanoemulsion

having droplet size of 60 Nanometer to 5 microns.

Bulk suspension preparation for rapidly disintegrating tablets process:

(i) preparing a complex suspension comprising Cannabidiol Oil in water emulsion, an oleaginous carrier, binder(s), matrix-forming agent(a) and pharmaceutically acceptable ingredients to obtain a homogenous suspension,

(ii) transferring the homogenous suspension to a dosing tank and stirring/homogenizing the said suspension until the end of the filling process

(iii) Forming blister pockets in blister filling machine. Use of peristaltic pump (or any pump of equivalent performance) to dose accurate amount of suspension into preformed blister pockets

(iv) freezing the drug suspension filled blister pockets by passing through liquid nitrogen freezing tunnel temperature ranging from −160° C. to −70° C. and pressure ranging from 3 kg/cm² to 9 kg/cm²,

(v) placing the frozen product into lyophilizer and lyophilizing the said frozen blister sheets at a primary drying temperature ranging from −30° C. to 0° C. followed by lyophilizing at a secondary drying temperature ranging from 15° C. to 25° C. under certain ranges of vacuum to obtain a rapidly disintegrating solid oral/sublingual dosage form comprising Cannabidiol.

The freeze-dried tablets thus obtained analyzed for complete finished product specification (Description, identification, assay, related substance, dissolution and water content).

The freeze-dried tablets disintegration resulted in 5 to 15 seconds (limit NMT 30 seconds).

### EXAMPLE—8

CBD Nanoemulsion Sublingual Tablet 10 mg

Qualitative and Quantitative composition CBD RDT 20 mg

| S. No. | Ingredients | Functional category | % Composition |
|---|---|---|---|
| 01 | Cannabidiol | Active | 1.0 to 10.0 |
| 02 | Polysorbate 80 | Emulsifying agent | 0.5-4.00 |
| 03 | Lecithin | Emulsifier | 0.5 to 2.0 |
| 04 | Purified water | Carrier vehicle | 80-90 |

Bulk suspension preparation

| | | | |
|---|---|---|---|
| 05 | Nano emulsion | | 5.00-15.00 |
| 06 | Beta cyclodextrin | Solubilizing agent | 5-20 |
| 07 | Microcrystalline | Diluent/binding agent | 1-2 |
| | cellulose | | |
| 08 | Lactose monohydrate | Bulking agent | 4-8 |
| 19 | Pototata starch and | Adhesive agents | 2-5 |
| | Amylopectin | | |
| 10 | Glycine | Emollient/emulsifying | 0.25-1 |
| | | agent | |
| 11 | Macrogol | Surfactant polymer | 0.25-1 |
| 12 | Pullulan | Matrix forming agent | 2.00-5.00 |
| 13 | Mannitol | Structure forming agent | 5.00-20.00 |
| 14 | Aspartame | Sweetener | 0.50-2.00 |
| 15 | Flavour | Flavouring agent | 0.1-3.00 |
| 16 | Purified water * | Carrier (Vehicle) | 40.00-60.00 |

* Water was removed during lyophilization processing

Manufacturing Process:

Nano Emulsion Preparation:

Oil in Water Emulsion Preparation Procedure.

Polysorbate 80 and Cannabidiol oil were taken in a separate beaker and subjected to sonification using ultrasonicator. This mixture was slowly added to the Lecithin/water pre-mix placed on water bath under sonification for 10 minutes at 30%-90% amplitude or high-pressure homogenizer to form a Nanoemulsion having droplet size of 60 Nanometer to 5 microns.

Bulk suspension preparation for lyophilized sublingual dispersion tablet process:

(i) Preparing the solution of Potato starch and Amylopectin, an adhesive agent by adding in purified water and mixing thoroughly with a stirrer. Then heating the mixture to 40° C. to 50° C. for 10 to 30 minutes to allow dissolving of the polymers.

(ii) Once the solution cooled down to room temperature, adding a surfactant polymer, emulsifying agent, diluent, bulking agent, matrix-forming agent and structure-forming agent i.e. Macrogol, glycine, microcrystalline cellulose, lactose, Pullulan and mannitol individually, under stirring to obtain a homogenous solution.

(iii) preparing a complex suspension comprising Cannabidiol Oil in water emulsion, an oleaginous carrier, binder(s), matrix-forming agent(a) and pharmaceutically acceptable ingredients to obtain a homogenous suspension,

(iv) transferring the homogenous suspension to a dosing tank and stirring/homogenizing the said suspension until the end of the filling process

(v) Forming blister pockets in blister filling machine. Use of peristaltic pump (or any pump of equivalent performance) to dose accurate amount of suspension into preformed blister pockets

(vi) freezing the drug suspension filled blister pockets by passing through liquid nitrogen freezing tunnel temperature ranging from −160° C. to −70° C. and pressure ranging from 3 kg/cm² to 9 kg/cm²,

(vii) placing the frozen product into lyophilizer. Lyophilizing the said frozen blister sheets at a primary drying temperature ranging from −30° C. to 0° C. followed by lyophilizing at a secondary drying temperature ranging from 15° C. to 25° C. under certain ranges of vacuum to obtain a rapidly disintegrating solid oral/sublingual dosage form comprising Cannabidiol.

(viii) The freeze-dried tablets analyzed for complete finished product specification (Description, identification, assay, related substance, dissolution and water content).

(ix) The freeze-dried tablets disintegration resulted in 5 to 15 seconds (limit NMT 30 seconds).

Examples as above are not limiting the scope of the invention. The quantities of the ingredients and the process steps thereof may be modified suitably to meet the lyophilizing or freeze-drying parameters and the consistency of the final suspension or solution being filled into the blister cavities for freeze drying and sealing.

We claim:

1. A pharmaceutical composition comprising a) cannabinoid in a concentration ranging from 1% to 50% w/w, b) one or more binding agent in a concentration ranging from 0.2% to 12% w/w, c) structure forming agent in a concentration ranging from 2% to 10% w/w and d) emulsifying agent or oleaginous vehicle in a concentration ranging from 0.1% to 20%.

2. The composition as claimed in claim 1, wherein the composition optionally comprise a solubilizing agent in a concentration ranging from 0 to 25%.

3. The composition as claimed in claim 1, wherein the composition is in the form selected from sublingual tablet, buccal tablet, and rapidly disintegrating tablet.

4. The composition as claimed in claim 1, wherein, the cannabinoid is selected from synthetic, semi synthetic or natural cannabinoid and extract of a cannabis plant, derivatives of cannabinoids and combination of cannabis plant constituents.

5. The composition as claimed in claim 1, wherein the cannabinoid is selected from the group consisting of Cannabidiol (CBD), delta-9-tetrahydrocannabinol (THC), Cannabinol (CBN), Cannabigerol (CBG), Cannabichromene (CBC), Cannabicyclol (CBL), Cannabivarin (CBV), Tetrahydrocannabivarin (THCV), Cannabidivarin (CBVD), Cannabichromevarin (CBVC), Cannabigerovarin (CBVG), Cannabigerol monoethyl ether (CBGM), a derivative, an acid form, a precursor, and a combination thereof.

6. The composition as claimed in claim 1, wherein, the binding agent is selected from the group consisting of maltodextrin, hypromellose, pullulan, fish gelatine, sodium alginate, xanthan gum.

7. The composition as claimed in claim 1, wherein, the emulsifying agent is selected from the group comprising of olive oil, sesame oil, castor oil, coconut oil, corn oil, lecithin, isolecithin, polysorbate 20 or polysorbate 80.

8. The composition as claimed in claim 1, wherein, the structure forming agent is selected from mannitol or dextran.

9. The composition as claimed in claim 1, wherein, the composition optionally comprise solubilizing agent selected from betacyclodextrin, Hydroxypropyl Beta-cyclodextrin or sufobutyl betacyclodextrin.

10. A process for preparation of rapidly disintegrating freeze dried solid oral tablet composition of cannabinoid, as claimed in claim 1, comprising the following steps:
(i) preparing a suspension comprising cannabidiol, emulsifying agent, matrix forming agent, structure-forming agent solubilizing agent and other pharmaceutically acceptable ingredients, in a solvent to obtain a homogenous suspension,
(ii) transferring the homogenous suspension to a dosing tank and stirring/homogenising the said suspension until the end of the filling process,
(iii) Forming blister pockets in blister filling machine,
(iv) Use of peristaltic pump (or any pump of equivalent performance) to dose accurate amount of suspension into preformed blister pockets,
(v) freezing the drug suspension filled blister pockets by passing through liquid nitrogen freezing tunnel temperature ranging from −160° C. to −70° C. and pressure ranging from 3 kg/cm2 to 9 kg/cm2 to obtain frozen products,
(vi) placing the frozen products into lyophilizer,
(vii) lyophilizing the said frozen blister sheets at a primary drying temperature ranging from −30° C. to −0° C. followed by lyophilizing at a secondary drying temperature ranging from 15° C. to 40° C. to obtain a rapidly disintegrating solid oral tablet composition comprising cannabidiol.

11. The process as claimed in claim 10, wherein, the cannabinoid is selected from the group consisting of Cannabidiol (CBD), delta-9-tetrahydrocannabinol (THC), Cannabinol (CBN), Cannabigerol (CBG), Cannabichromene (CBC), Cannabicyclol (CBL), Cannabivarin (CBV), Tetrahydrocannabivarin (THCV), Cannabidivarin (CBVD), Cannabichromevarin (CBVC), Cannabigerovarin (CBVG), Cannabigerol monoethyl ether (CBGM), a derivative, an acid form, a precursor, and a combination thereof.

12. The process as claimed in claim 10, wherein, the binding agent is selected from the group consisting of maltodextrin, hypromellose, pullulan, fish gelatine, sodium alginate, xanthan gum; the oleaginous vehicle/emulsifying agent/dispersing agent is selected from the group comprising of olive oil, sesame oil, castor oil, coconut oil, corn oil, lecithin, isolecithin, polysorbate 20 or polysorbate 80; the structure forming agent is selected from mannitol or dextran and the solubilizing agent selected from betacyclodextrin, Hydroxypropyl Beta-cyclodextrin or sufobutyl betacyclodextrin.
